(19) United States
(12) Patent Application Publication
(10) Pub. No.: US 2006/0162730 A1
(43) Pub. Date: Jul. 27, 2006

(54) Video-assisted laryngeal mask airway devices

(76) Inventors: Raymond Glassenberg, Wilmette, IL (US); Zebadiah Kimmel, Brookline, MA (US)

Correspondence Address: LUCE, FORWARD, HAMILTON & SCRIPPS LLP, 11988 EL CAMINO REAL, SUITE 200, SAN DIEGO, CA, 92130, US

(21) Appl. No.: 11/044,559

(22) Filed: Jan. 26, 2005

(51) Int. Cl. A62B 9/06; A61M 16/00

(52) U.S. Cl. 128/207.14; 128/207.15; 128/200.26

(57) ABSTRACT

A laryngeal mask airway device is provided that incorporates a video sensor, such as a CCD, CMOS or NMOS imaging chip, arranged to provide an image of the laryngeal inlet or other airway structures. The video sensor is electrically coupled to a reusable processing unit that receives the signals generated by the video sensor and generates a digital image of the interior of the patient's airway, thereby enabling the clinician to have immediate optical confirmation of the position of the mask aperture relative to the laryngeal inlet from the moment of insertion of the device.

### FIELD OF THE INVENTION

The present invention relates to laryngeal mask airway devices, such as laryngeal mask airways and intubating laryngeal masks, for use in administering anesthesia having one or more video sensors mounted in the bowl of the device to assist in placement of the device or insertion of an endotracheal tube.

### BACKGROUND OF THE INVENTION

Laryngeal mask airways (“LMA”) are known for use in administering anesthesia in lieu of, or in conjunction with endotracheal tubes. LMAs permit ventilation of the patient without placing an endotracheal tube into the trachea, but do not protect against the risks of regurgitation and aspiration. Commercially available LMAs are designed to reduce the risk encountered with endotracheal tubes of improper placement of the tube in the esophagus rather than then trachea, and are now are used in more than ⅓ of all anesthetic procedures. Such devices generally include a flexible tube that is coupled to and communicates with a mask part comprising a bowl surrounded by an inflatable cuff. The device may be blindly inserted into the pharynx and when so positioned, the mask part seals around the glottis.

Despite the general success of LMAs, intubation of the trachea often remains a key aspect of airway management, such as in an emergency or when there may be a risk of aspiration of gastric contents, since the presence of a cuffed tube in the trachea prevents gastric acid present in vomit from entering and damaging the lungs. However, intubation of the trachea is not always possible and, when difficulty is experienced, soiling of the lungs with gastric acid may occur while attempts are being made to intubate. In cases where intubation by conventional means, such as using a laryngoscope to visualize the glottis has failed, a modified form of the LMA may be used as a guide to facilitate intubation. The LMA-Fastrach™, distributed by LMA North America, San Diego, Calif., is such as device, and is generally referred to as an “intubating laryngeal mask” (“ILM”).

ILMs have the limitation that, for a high degree of success in passing an endotracheal tube through the ILM tube into the trachea, fiberscopic aid is needed to ensure the endotracheal tube does not pass into the esophagus or collide with and injure the epiglottis. These hazards, particularly the former, which may result in death if undetected, are similar to those encountered in classical intubation using a laryngoscope. Fiberoptic assisted intubation, where a fiberscope is used to visualize placement of the ILM and endotracheal tube, may be employed when classical intubation fails. However, fiberoptic assisted intubation has the disadvantage that it requires considerable skill and time, significant drawbacks in cases where brain damage or death from lack of oxygen are imminent if ventilation cannot be achieved.

Advantageously, LMAs and ILMs (collectively “LMA devices”) permit a patient to be kept alive even where intubation turns out not to be impossible because, unlike the laryngoscope or the fiberoptic scope (“fiberscope”), the mask part of the LMA device provides an adequate seal around the glottis to permit gentle positive pressure ventilation to be maintained while intubation attempts are ongoing. This is a critical advantage compared to prior art techniques because death or brain damage more often occur from failure to ventilate the lungs than from lung contamination with gastric contents.

In fiberoptic assisted intubation, the clinician reaches the laryngeal aperture by passing the fiberscope around the back of the tongue (or through the nasal cavity and nasopharynx) and then passing the tip of the scope downwards until the larynx comes into view. Insertion of the fiberscope in this manner takes time and skill. Because the scope typically has a small cross-section relative to the cross-section of the pharynx, it is possible for the tip of the fiberscope to wander to one side or the other of the pharynx during insertion, and thus miss the structures of the laryngeal orifice.

In addition, the tip of the fiberscope is not protected from contamination with secretions present in the pharynx or from bleeding provoked by its passage, either or both of which may obscure the fiberscope operator's view. Moreover, a further problem encountered with fiberoptic assisted intubation is that the view is two-dimensional and the field of vision is very restricted. The combination of all these factors makes fiberoptic assisted intubation a difficult skill to acquire and maintain. Lastly, fiberscopes are very expensive and not all hospitals are able to afford or maintain them, thereby adding to the difficulty of ensuring that clinicians have the necessary skill to use the technique.

The foregoing problems are partly resolved when the LMA device is used as a guide for the fiberscope, since when correctly inserted, the mask part of the LMA device completely fills the space of the lower pharynx when the cuff surrounding the mask is deployed. Time to first ventilation is very rapid as the device may be passed blindly in a single movement. Accordingly, when using a LMA device, a view of the laryngeal inlet is automatically achieved in the great majority of cases simply by inserting the fiberscope down the tube of the LMA device, wherein the LMA device acts as a guide directing the fiberscope to its target. One such method is described in U.S. Pat. No. 5,623,921 to Kinsinger et al. Once a LMA is placed in the patient's pharynx and the fiberscope is disposed in the tube of the LMA, inspection may be carried out in an unhurried manner, since adequate ventilation is assured as soon as the LMA device is deployed. With commercially-available ILMs, the probability of viewing the larynx is even greater because the ILM tube is rigid and provided with an external handle that permits direct manipulation of the mask relative to the larynx, thereby allowing the clinician to alter the position of the mask if perfect alignment is not achieved during blind insertion. However, a fiberscope still has to be inserted in the tube to ascertain whether accurate alignment has been achieved.

U.S. Pat. No. 5,682,880 to Brain describes a LMA having a passageway that accepts a removable stiffening member, which may be used to install the LMA. The patent describes that once the LMA is placed, the stiffening member is removed from the passageway. An optical fiber then is inserted into the passageway to visualize the laryngeal inlet and facilitate endotracheal tube insertion. European Patent EP 0 768 903 B1 to Brain also describes an ILM including a passageway that accepts an optical fiber to facilitate endotracheal tube placement.

Recent studies have indicated that direct visualization also may be useful in improving placement of an LMA over the conventional blind insertion method. Campbell et al., Fiberoptic Assessment of Laryngeal Mask Airway Placement: Blind Versus Direct Visual Epiglottoscopy, J. Oral Maxillofac. Surg. 2004 September; 62(9)1108-1113, describes use of a fiberscope to compare LMA placement performed using a laryngoscope (direct visualization) to blind placement. The article observed that ideal placement was obtained in more than 90% of the cases where a laryngoscope was used, as compared to only 42% of the blind placement cases.

Further still, recent studies have shown the injury to the laryngeal nerve may be substantially reduced during thyroid surgery by visualizing the laryngeal nerve using a fiberscope placed through the airway tube of an LMA. The results of two such studies are described in M. C. Scheuller and D. Ellison, Laryngeal Mask Anesthesia With Intraoperative Laryngoscopy for Identification of the Recurrent Laryngeal Nerve During Thyroidectomy, Laryngoscope, 112:1594-1597 (2002) and H. K. Eltzschig et al., The Use of Readily Available Equipment in a Simple Method for Intraoperative Monitoring of Recurrent Laryngeal Nerve Function During Thyroid Surgery Initial Experience With More Than 300 Cases, Arch. Surg., 137:452-457 (2002).

In view of the foregoing, there is a recognized need for visualization aids to improve placement of LMAs and endotracheal tubes, and to improve visualization of the patient's airway during airway-related surgical procedures. Although the foregoing patents to Brain disclose LMA devices that include fiberoptic components to enhance viewing, there are several disadvantages to the use of optical fibers. Generally, such fibers are susceptible to breakage during bending, require a high degree of illumination, and are susceptible to image distortion as the reflected light travels through the optical fiber. In addition, the electronics components required to process and display an image transmitted through an optical fiber are expensive, thereby limiting acceptance of such devices.

In recognition of these drawbacks of the previously-known fiberoptic systems, some previously known devices have attempted to incorporate a video camera, such as a charge-coupled device (“CCD”), at the distal end of the device to provide improved visualization. Hill U.S. patent application publication US2003/0078476 describes an endotracheal tube having CCD camera disposed at its distal end. U.S. Pat. No. 6,652,453 to Smith et al. and U.S. Pat. No. 5,827,178 to Berall each disclose a laryngoscope having a camera mounted in the vicinity on the distal end that generates an image displayed on a screen of the device. However, all of these devices suffer from the disadvantage noted above. Specifically, none of these devices provide an adequate degree of ventilation to the patient while the intubation process is underway.

In view of the foregoing, it would be desirable to provide an LMA device, configured as either a LMA or an ILM, that includes a video sensor disposed in mask portion of the device to provide visualization of the laryngeal inlet and other airway structures.

It also would be desirable to provide single-use LMA devices that incorporate low-cost, solid state camera components, such as a CCD, CMOS or NMOS sensor, that may be coupled to a reusable processing unit and display screen.

It further would be desirable to provide LMA devices having two or more video sensors with intersecting fields of view, thereby enabling the clinician to obtain a stereoscopic view of the patient's airway.

It still further would be desirable to provide LMA devices wherein the inflatable cuff is arranged to be self-expanding, thereby obviating the need for the clinician to separately attend to inflating the cuff during placement of the LMA device.

### SUMMARY OF THE INVENTION

In of the foregoing, it is an object of the present invention to provide an LMA device, configures as either LMA or ILM, that includes a video sensor disposed in mask or bowl portion of the device to provide visualization of the laryngeal inlet and other airway structures.

It is also an object of this invention to provide to provide single-use LMA devices that incorporate low-cost, solid state camera components, such as a CCD, CMOS or NMOS video sensor and an illumination source, such as a light emitting diode (“LED”), that may be coupled to a reusable processing unit and display screen.

It is another object of the present invention to provide LMA devices having two or more video sensors with intersecting fields of view, thereby enabling the clinician to obtain a stereoscopic view of the patient's airway.

It is a further object of this invention to provide LMA devices wherein the inflatable cuff is arranged to be self-expanding, thereby obviating the need for the clinician to separately attend to inflating the cuff during placement of the LMA device.

These and other objects of the present invention are accomplished by providing a LMA device, configured as either an LMA or ILM, that incorporates a video sensor, such as a CCD, CMOS or NMOS sensor, arranged to provide an image of the laryngeal inlet and/or other airway structures. In this manner, the LMA device of the present invention permits the clinician to have immediate optical confirmation of the position of the mask aperture relative to the laryngeal inlet from the moment of insertion of the device and at any time thereafter. In the case of an intubating laryngeal mask, the video sensor permits image-guided intubation using a conventional endotracheal tube. In one preferred embodiment, the LMA device may include two or more video sensors having intersecting fields of view, thereby providing a stereoscopic view of the patient's airway.

In accordance with one aspect of the present invention, the LMA device is disposable and discarded after a single-use. The video sensor of the LMA device includes electrical lead wires that terminate in a connector that may be coupled to a reusable unit that processes the signals from the video sensor to generate digital images. The LMA additionally may include an illumination system, such as an LED, to provide lighting within the patient's airway. Preferably, the LMA device may be coupled to a reusable module that houses electronics for powering the video sensor, processing the signals generated by the video sensor, and optionally, powering the illumination system. The reusable module also may include a screen for displaying the images generated by the video system, or may input an output suitable for display on a conventional display.

In accordance with another aspect of the present invention, the cuff disposed surrounding the mask portion of the LMA device comprises an open-cell foam disposed in a fluid impermeable plastic cuff. The open-cell foam may be evacuated to mechanically compress the foam and then retained in the compressed state by reversibly sealing the cuff. As opposed to conventional LMA devices, wherein the cuff is inflated by injecting air into the cuff using a syringe, the cuff of the LMA device of the present invention may be deployed simply by unsealing a lumen connected to the cuff. In this manner, the open-cell foam will automatically expand to conform to seal around the patient's glottis.

### BRIEF DESCRIPTION OF THE DRAWINGS

The above and other objects and advantages of the present invention will be apparent upon consideration of the following detailed description, taken in conjunction with the accompanying drawings, in which like reference numerals refer to like parts throughout, and in which:

FIG. 1 is a side view, partly schematic, of a LMA constructed in accordance with the principles of the present invention;

FIGS. 2A and 2B are, respectively, a view along line 2A-2A in FIG. 1 and a perspective view of the mask portion of the device of FIG. 1;

FIG. 3 is a cross-sectional side view of the mask portion of the device of FIG. 1;

FIGS. 4A and 4B are perspective views of the mask portion of the device of FIG. 1 wherein the cuff is shown in the deployed and delivery configurations, respectively;

FIG. 5 is a side view showing the device of FIG. 1 inserted into a patient's airway;

FIG. 6 is a perspective view of an intubating laryngeal mask constructed in accordance with the principles of the present invention; and

FIG. 7 is a side view showing the device of FIG. 6 inserted into a patient's airway.

### DETAILED DESCRIPTION OF THE INVENTION

In accordance with the principles of the present invention, a video laryngeal mask airway (“LMA”) device is provided to facilitate lung ventilation in an unconscious patient, comprising an airway tube and a mask attached to an end of the airway tube. The mask communicates with the airway tube and includes a peripheral cuff having a roughly elliptical shape that is configured to conform to and readily fit within the space behind the larynx. In this manner, the cuff forms a seal around the circumference of the laryngeal inlet without the device penetrating into the interior of the larynx. In accordance with the present invention, the mask carries at least one video sensor having a field of view that encompasses the laryngeal inlet when the mask is inserted into the patient's airway. The LMA device, which may be configured as either an LMA or ILM, preferably is disposed of after a single use. Alternatively, the LMA device may have the video sensors oriented within the mask portion so as to provide a desired view of other airway structures, such as the vocal cords.

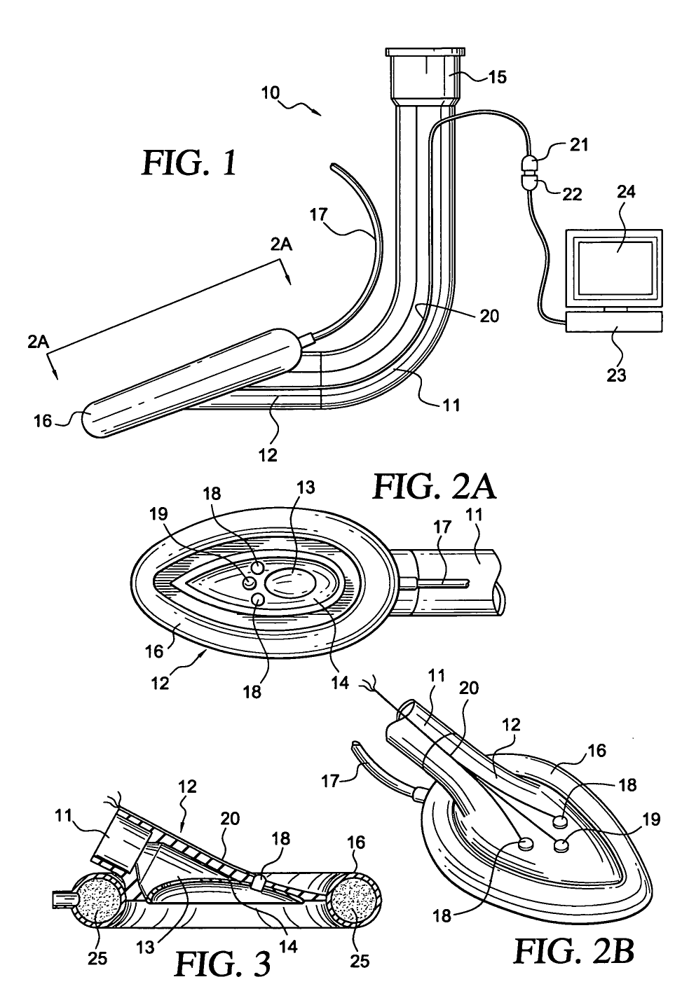

Referring to FIGS. 1-3, an exemplary LMA device constructed in accordance with present invention is described. LMA device 10, illustratively a laryngeal mask airway, includes flexible airway tube 11 coupled to mask portion 12.

As is conventional, airway tube 11 is curved and pliable to follow the airway of the patient, and communicates with opening 13 in the bowl-shaped lower surface 14 of mask portion 12. Airway tube 11 includes connector 15 for coupling the tube to a ventilation device. Mask portion 12 includes cuff 16 disposed along the periphery of the mask portion, which has a roughly elliptical shape. Cuff 16 comprises an elastomeric material and includes tubing 17 that permits the cuff to be contracted for insertion or deployed by removing or adding air. Cuff 16 is configured to conform to and readily fit within the space behind the larynx, and thereby form a seal around the circumference of the laryngeal inlet.

LMA device 10 further includes at least one video sensor 18, preferably either a charge-coupled device (CCD) such as are used in digital video cameras or CMOS or NMOS image sensor. Video sensor 18 may be fabricated using any of a number of semiconductor chip manufacturing processes. Video sensor 18 is mounted in mask portion 12 and directed so that its field of vision is aligned with opening 13 and encompasses the laryngeal inlet or other desired airway structure when the LMA device is inserted into a patient's throat. Optionally, mask portion 12 also may include illumination source 19, such as a light emitting diode (LED), to illuminate the patient's airway during placement of LMA device 10 and deployment of cuff 16.

In the illustrative embodiment depicted in FIGS. 1-3, mask portion 12 includes two video sensors 18 having illumination source 19 disposed therebetween. Advantageously, video sensors 18 are directed so that their fields of view overlap, thereby providing the clinician with a stereoscopic view of the patient's anatomy. As depicted in FIG. 3, each video sensor 18 preferably is embedded or potted in the wall of mask portion 12 and comprises a CCD, CMOS or NMOS chip disposed in plastic housing with an optically clear window. It is to be understood that the use of only a single video sensor is within the scope of the present invention, and that positioning of a single video sensor within the mask portion may be selected to optimize the field of view provided by the sensor.

Video sensors 18 and illumination source 19 are coupled via electrical leads 20 that terminate in connector 21. Electrical leads 20 are disposed within a non-conductive tube affixed to an exterior surface of airway tube 11, or alternatively, may be disposed within an interior lumen in the wall of airway tube 11. Connector 21 may be coupled to mating connector 22, which in turn is coupled to processing unit 23 and display screen 24.

Processing unit 23 supplies power to video sensors 18 and illumination source 19 and converts the signals generated by video sensors 18 into a video image that may be displayed on screen 24. In this manner, the clinician may insert the LMA device guided by the video supplied from video sensors 18 to processing unit 23 and display 24, thereby attaining optimum placement of the mask portion 12 of the LMA device. Processing units 23 for powering a video sensor and converting the output of such a sensor to a video image are known in the art, and may be of the type commonly used in digital video camcorders. Display screen 24 may comprise any suitable video display and may be either integral with, or separate from, processing unit 23. Alternatively, the LMA device may include an on-board power source, such as a battery, conveniently located on the airway tube or on the mask portion of the LMA device to power the video sensors or illumination source. In this latter case, the processing unit need only receive the signal output by the video sensor and convert that data to a digital image for display on screen 24.

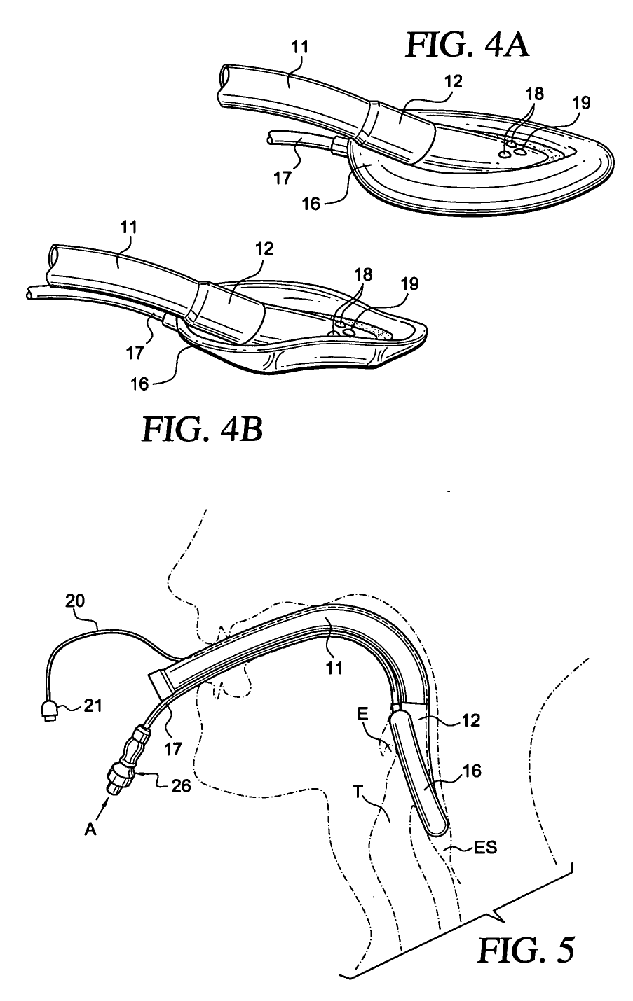

Referring now to FIGS. 3 and 4, cuff 16 may be of conventional construction and comprise an elastomeric material that is deployed by inflation using a pressurized gas (e.g., air) or fluid. In a preferred embodiment, however, cuff 16 is filled with open-cell foam 25 that may be compressed to a small volume when evacuated (FIG. 4B) and that re-expands to conform to and seal around the laryngeal inlet in when deployed (FIG. 4A). One preferred material for open-cell foam 25 is an open-cell polyurethane foam.

Referring now also to FIG. 5, in operation cuff 16 is compressed to drive the air out of the foam via tubing 17 and the tubing is then sealed using removable plug 26. Cuff 16 also may be folded upwards around mask portion when compressed, as depicted in FIG. 4B, so that the periphery of the mask does not impede insertion of LMA device. Mask portion 12 then is inserted through the patient's mouth and disposed just above the patient's esophagus ES so that opening 13 of mask portion 12 is disposed below epiglottis E and in alignment with the patient's laryngeal inlet, as determined by video guidance using video sensors 18. Once the LMA device is seated surrounding the laryngeal inlet, plug 26 is opened to permit air to flow into tubing 17, as indicated by arrow A. This in turn allows foam 25 to re-expand to seal around the laryngeal inlet, permit ventilation and prevent inhalation of gastric fluids into the patient's lungs, as depicted in FIG. 4A.

The LMA device of the present invention permits immediate optical confirmation of the position of the mask, which in turn provides at least the following additional advantages:
The presence of regurgitant fluid in the bowl of mask portion 12, before intubation of the trachea is completed, may immediately be seen and aspirated using a suction catheter before significant lung contamination occurs.
Visual information from the video sensors may be transferred to a television screen for remote viewing, for example, as part of the monitoring equipment on the anesthetic machine.
Video images provided by the video sensors may be stored for future use in teaching or as part of the patient's case notes, for example for medico-legal evidence.
Laryngeal movements indicating inadequate levels of anesthesia may be observed, thereby permitting early intervention to reduce the danger of laryngeal spasm or awareness.
Laryngeal movement resulting from electrical stimulation may be readily monitored to preserve laryngeal nerve function.
Like a previously-known LMA, the device may be inserted in an awake patient after application of local anesthesia to the throat, thereby offering the possibility of treatment and diagnosis of upper airway problems on an outpatient basis.

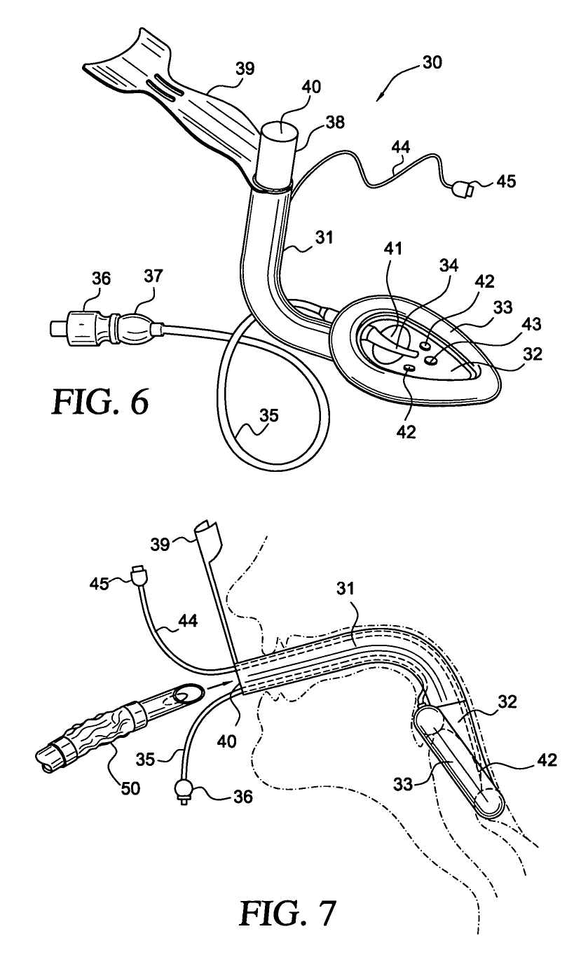

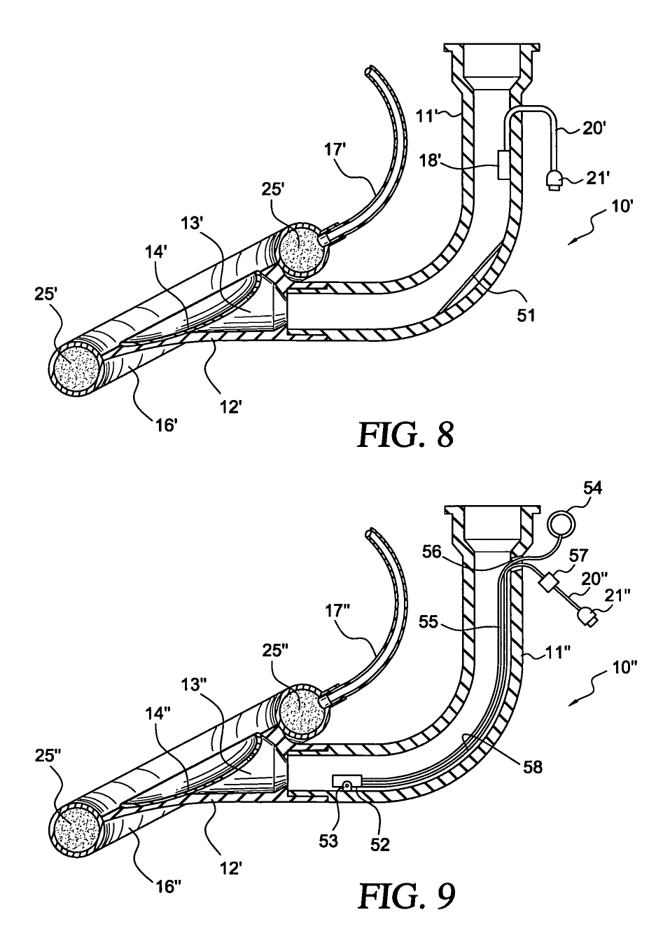

Referring now to FIGS. 6 and 7, an alternative embodiment of the LMA device of the present invention is described, illustratively an intubating laryngeal mask (“ILM”). The ILM depicted in FIG. 6 is similar in design to that commercially marketed by LMA North America, Inc., under the trade-name “LMA-Fastrach™” and comprises curved airway tube 31 attached to mask portion 32. Mask portion 32 is surrounded by generally elliptical cuff 33 at its periphery. Mask portion 32 and cuff 33 are of conventional construction and configuration, such as described above, and optionally may include epiglottis elevating bar 34. Pressurized gas is supplied to and withdrawn from cuff 33 using tubing 35 via valve 36 and pilot balloon 37.

Airway tube 31 comprises a pliable plastic coating disposed over metal tube 38 that extends from external rigid handle 39 to the bowl of mask portion 32. The airway tube includes main airway lumen 40 that communicates with the bowl of mask portion 32 via opening 41. Handle 39 extends is used to position and manipulate the ILM in the patient's throat. Airway tube 31 is provided with easily removable friction-fit connector (not shown) designed for attachment to conventional anesthetic gas hosing, so that the device may be used in a stand-alone manner to ventilate the lungs of a patient, without intubating the patient with an endotracheal tube.

In accordance with the principles of the present invention, ILM includes video sensors 42 and illumination source 43 disposed in the bowl of mask portion 32. Video sensors 42 preferably comprise CCD, CMOS or NMOS devices, while illumination source 43 preferably comprises an LED, as described above. Video sensors 42 and illumination source 43 are coupled via electrical leads 44 to connector 45, which may be coupled to a processing unit so that signals generated by video sensors 42 may be converted to digital images and displayed on a display screen, such as described above with respect to FIG. 1.

Video sensors 42 preferably are disposed in the bowl of mask portion 32 close to opening 41 of the mask portion at such an angle as to offer a view of the larynx and more preferably, so that the field of vision of the video sensors overlap so as to provide a stereoscopic view of the larynx 11. In this manner, if intubation of the trachea with an endotracheal tube is desired, as depicted in FIG. 7, the laryngeal view from the video sensors may be used to help the clinician guide the tip of the endotracheal tube towards the laryngeal inlet. In addition, the ILM may be manipulated using handle 39 to improve alignment between opening 41 of mask portion 32 and the laryngeal inlet.

In FIG. 7, the ILM of FIG. 6 is shown disposed in a patient's throat with cuff 33 deployed so that mask portion 32 surrounds and seal the laryngeal inlet. Once the ILM is positioned as shown, the proximal end of the ILM may be intermittently coupled to a ventilation system to provide positive ventilation to the patient. If it is desired to intubate the patient with endotracheal tube 50, the gas hose from the ventilation system (not shown) may be removed, and endotracheal tube 50 inserted through lumen 40 of airway tube 31. Using the video images generated by video sensors 42, the clinician may then manipulate handle 39 to guide the tip of the endotracheal tube into the patient's trachea.

Advantageously, the features of the present invention may be incorporated into any form of laryngeal mask device and are not limited to the exemplary embodiments set forth above and it will be evident to one skilled in the art that various changes and modifications may be made therein without departing from the invention. It is intended in the appended claims to cover all such changes and modifications that fall within the true spirit and scope of the invention.

What is claimed is:

1. A laryngeal mask airway device to facilitate lung ventilation in a patient, comprising:
an airway tube having proximal and distal ends;
a mask portion affixed to the distal end of the airway tube;
a cuff having a roughly elliptical shape disposed around the periphery of the mask portion, the cuff configured to form a seal around a circumference of the patient's laryngeal inlet, without penetrating into an interior of the patient's larynx;
a first video sensor disposed within the mask portion, the first video sensor having a field of view that encompasses the patient's laryngeal inlet so as to provide visual confirmation of placement of the mask portion.

2. The laryngeal mask airway device of claim 1, further comprising an illumination source disposed within the mask portion to illuminate the patient's airway.

3. The laryngeal mask airway device of claim 1, further comprising a second video sensor disposed within the mask portion, the second video sensor having a field of view that overlaps the field of view of the first video sensor.

4. The laryngeal mask airway device of claim 1, wherein the device is intended for disposal after a single use.

5. The laryngeal mask airway device of claim 1, further comprising a reusable processing unit for converting signals received from the first video sensor into digital images.

6. The laryngeal mask airway device of claim 5, further comprising a display screen configured to be coupled to the processing unit to display digital images generated by the processing unit.

7. The laryngeal mask airway device of claim 1, wherein the first video sensor is a charge-coupled device or CMOS or NMOS device.

8. The laryngeal mask airway device of claim 2, wherein the illumination source is a light emitting diode.

9. The laryngeal mask airway device of claim 1, further comprising a rigid handle for manipulating the laryngeal mask airway device during placement of an endotracheal tube.

10. The laryngeal mask airway device of claim 1, further comprising a foam disposed within the cuff, the foam having a delivery state wherein the foam is compressed to a small volume when evacuated and a deployed state wherein the foam re-expands to conform to and seal around the laryngeal inlet.

11. A laryngeal mask airway device to facilitate lung ventilation in a patient, comprising:
an airway tube having a lumen and proximal and distal ends;
a mask portion affixed to the distal end of the airway tube, the mask portion having an opening that communicates with the lumen of the airway tube;
a cuff disposed around the periphery of the mask portion, the cuff having a contracted delivery state and a expanded deployed state wherein the cuff forms a seal around a circumference of the patient's laryngeal inlet;
a first video sensor affixed to the mask portion, the first video sensor having a desired field of view within the patient's airway after placement of the mask portion within the patient's airway.

12. The laryngeal mask airway device of claim 11, further comprising an illumination source affixed to the mask portion to illuminate the patient's airway.

13. The laryngeal mask airway device of claim 11, further comprising a second video sensor affixed within the mask portion, the second video sensor having a field of view that overlaps the field of view of the first video sensor.

14. The laryngeal mask airway device of claim 11, wherein the device is intended for disposal after a single use.

15. The laryngeal mask airway device of claim 11, further comprising a reusable processing unit for converting signals received from the first video sensor into digital images.

16. The laryngeal mask airway device of claim 15, further comprising a display screen configured to be coupled to the processing unit to display digital images generated by the processing unit.

17. The laryngeal mask airway device of claim 11, wherein the first video sensor is a charge-coupled device or CMOS or NMOS device.

18. The laryngeal mask airway device of claim 12, wherein the illumination source is a light emitting diode.

19. The laryngeal mask airway device of claim 11, further comprising a rigid handle for manipulating the laryngeal mask airway device during placement of an endotracheal tube.

20. The laryngeal mask airway device of claim 1, further comprising a foam disposed within the cuff, the foam having a delivery state wherein the foam is compressed to a small volume when evacuated and a deployed state wherein the foam re-expands to conform to and seal around the laryngeal inlet.
